(19) United States
(12) Patent Application Publication
(10) Pub. No.: US 2008/0075668 A1
(43) Pub. Date: Mar. 27, 2008

(54) Security Device Using Reversibly Self-Assembling Systems

(76) Inventor: Alan H. Goldstein, San Francisco, CA (US)

Correspondence Address: SHELDON MAK ROSE & ANDERSON PC, 100 East Corson Street, Third Floor, PASADENA, CA, 91103-3842, US

(21) Appl. No.: 11/535,875

(22) Filed: Sep. 27, 2006

(51) Int. Cl. A61K 9/44

(52) U.S. Cl. 424/9.6; 424/10.2; 106/31.03

(57) ABSTRACT

A security device having: a base having a pattern thereon; a mobile component disposed in contact with the base, the mobile component containing a plurality of reversibly adsorbable particles; and a cover attached to the base around the mobile component to maintain the mobile component in contact with the base; wherein the adsorbable particles are mobile and reversibly changeable between a first state where the adsorbable particles are adsorbed to at least a predetermined percentage of the pattern and a second state where the adsorbable particles are adsorbed to less than the predetermined percentage of the pattern.

### BACKGROUND

The present invention relates to the identification and authentication of goods as genuine products from counterfeit versions thereof. In particular, the invention relates to labels or features that may be affixed to or otherwise incorporated into genuine goods.

Counterfeiting documents and products, such as bank notes, checks, tickets, credit cards and the like, and valuable merchandise and items, is a common problem. To prevent counterfeiting, many secure documents and other items of value include one or more security devices disposed on or in the item. Security devices typically operate via one or more technical strategies, such as metallic security features, magnetic security features, or luminescent security features, that authenticate the document and prevent counterfeiting.

However, existing security devices often suffer from one or more of the following problems: they are easily circumvented by direct counterfeiting or simulation, are expensive to produce, have a limited lifetime, and require specialized and often expensive detection equipment. Thus, there is a need for an improved security device that overcomes the shortcomings of the prior art.

### SUMMARY

Accordingly, the present invention is directed to a security device with a base having a pattern thereon; a mobile component disposed in contact with the base, the mobile component containing a plurality of reversibly adsorbable particles; and a cover attached to the base around the mobile component to contain the mobile component in contact with the base.

The adsorbable particles are mobile and reversibly changeable between a first state where the adsorbable particles are adsorbed to at least a predetermined percentage of the pattern and a second state where the adsorbable particles are adsorbed to less than the predetermined percentage of the pattern. Preferably, the particles are reversibly changeable through molecular self assembly. Optionally, when the adsorbable particles are in the first state, the pattern can be visually detected by an unaided human eye.

The adsorbable particles may have a dye. The base may have a lip around an outer edge with the cover being attached to the lip. The base may also have a protective layer, with a substrate attached to the protective layer, the pattern being formed on the substrate. Optionally, the protective layer may also be made of silicone rubber or silicone elastomer. The cover may be a polyimide film or a fluoropolymer film.

The adsorbable particles may change from the first state to the second state and back to the first state in less than 5 seconds. Additionally, the adsorbable particles may change from the first state to the second state and back to the first state more than 10,000 times. The device may operate in a temperature range of from about −20 degrees Celsius to about 70 degrees Celsius.

The present invention is also directed to a method for making a security device comprising: forming a base with a pattern; coupling the base to a cover; injecting a mobile component between the base and the pattern through the cover, the mobile component comprising a plurality of adsorbable particles; and sealing the cover. Additionally, forming the base may further comprise: depositing a pattern material on a substrate in a pattern; and attaching the substrate to a protective layer.

The device of the present invention may be affixed to or incorporated into documents or products, such as currency, driver's licenses, passports and purses or other consumer goods. In one embodiment of the device, a pattern or image in the device would be visible to the unaided human eye. However, by applying energy to the device, such as by depressing it with a human finger, the pattern or image would disappear for a short period of time (such as five seconds) and then reappear. Thus, the user could authenticate the document or product as genuine by viewing the disassembly and reassembly of the pattern or image. For additional security, the device could also contain a pattern or image that is not visible to the human eye and may only be detected using an appropriate machine. Additionally, forensic features can be created by adding an additional pattern and complementary chemistry whose detection method is known only to the manufacturer and security-cleared users.

### BRIEF DESCRIPTION OF THE DRAWINGS

A better understanding of the present invention will be had with reference to the accompanying drawings in which:

FIG. 1 is a schematic side view of a device according to an embodiment of the present invention in a first state where at least a portion of the adsorbable particles are adsorbed to the pattern on the base;

FIG. 2 is a schematic side view of the device of FIG. 1 in a second state where the majority of the adsorbable particles are not adsorbed to the pattern;

FIG. 3 is a schematic top view of the device of FIG. 1;

FIG. 4 is a schematic top view of the device of FIG. 2;

FIG. 5 is a schematic side view of a device according to an additional embodiment of the present invention having a protective layer in a first state where at least a portion of the adsorbable particles are absorbed to the pattern on the base; and

FIG. 6 is a schematic side view of the device according to FIG. 5 where the cover has been compressed and the majority of the adsorbable particles are not adsorbed to the pattern.

### DETAILED DESCRIPTION

As used herein the term “particle” refers to a mobile entity ranging in size from an atom to mesoscale metallic particles or colloids.

As used herein the term “adsorbable” refers to the capacity of a particle to attach to a pattern on a substrate. The adsorption may be physisorption or chemisorption such that the energy of binding is low enough for the adsorption to be reversible to create cycles of assembly, disassembly and reassembly (“ADR cycles”).

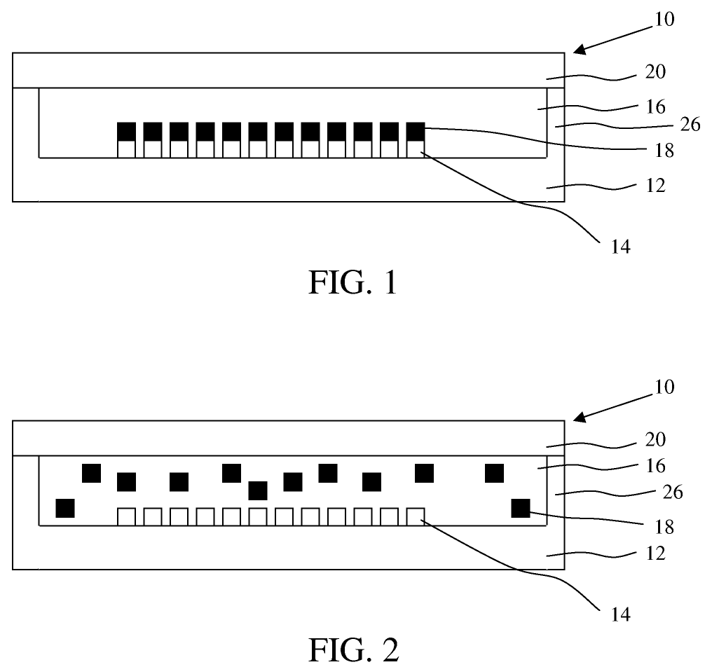

As shown in FIGS. 1 to 4, the present invention, according to an embodiment, is directed to an identification and security device 10 that uses reversibly self-assembling molecular surface structures to create a detectable image. The device 10 has a base 12. The base has a pattern 14 formed thereon. Preferably, a different material is micro-patterned or nano-patterned onto the base to form the pattern 14. A mobile component 16 covers the base 12 and the pattern 14.

The mobile component 16 contains a plurality of adsorbable particles 18 that reversibly adsorb to the pattern 14 on the base 12, but not to the remainder of the base. This creates a high resolution image when the adsorbable particles selectively adsorb to the pattern. Reversibility is based on the quasi-equilibrium nature of the adsorption process whereby the input of relatively small amounts of energy (often as low as 1-5 kcal/mole) will result in desorption, and therefore, disassembly, of the self-assembled molecular surface structure. A cover 20 encompasses and seals the mobile component 16 in contact with the base 12 and the pattern 14.

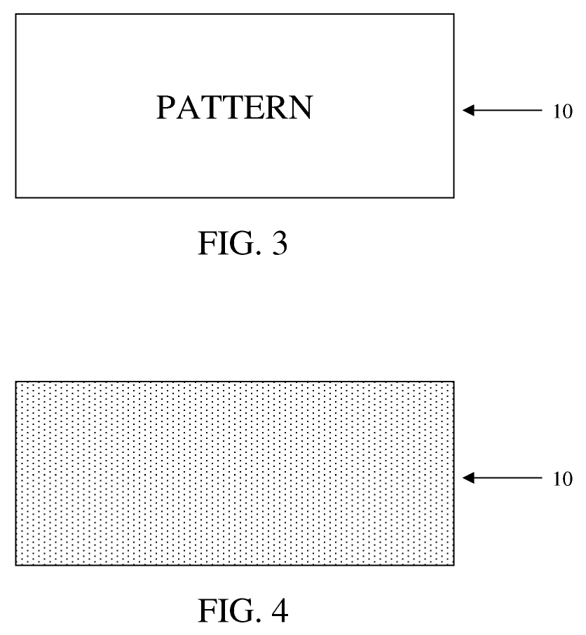

The device forms a closed thermodynamic system. The device 10 can undergo repeated cycles of assembly, disassembly and reassembly. During assembly, as shown in FIGS. 1 and 3, the adsorbable particles 18 adsorb (through molecular self assembly) to the material of the pattern 14 on the base 12 to form a detectable image. The chemistry of the base is selected so that the base does not adsorb the adsorbable particles. During disassembly, as shown in FIGS. 2 and 4, the adsorbable particles 18 detach from the pattern 14, which in turn causes the loss of the detectable image. During reassembly, the adsorbable particles 18 re-adsorb to the pattern through molecular self assembly, thereby again forming the detectable image shown in FIG. 3.

In an alternative embodiment of the present invention, the pattern 14 may be detectable, for example, by an unaided human eye, when no adsorption particles 18 are adsorbed thereto and substantially undetectable when the adsorption particles are adsorbed to the surface of the pattern.

In another alternative embodiment, the adsorbable particles 18 may change from the first state to the second state a limited number of times, and then the adsorbable particles would permanently change state so that ADR cycling no longer occurs. Chemical degradation of the adsorbable particles 18 may result in permanent loss of pattern detection. Alternatively, oxidation or another process could change the energetics of adsorption so that the adsorbable particles 18 bind to the patterned surface 14 with enough energy so that ADR cycling cannot occur, resulting in a permanent visible pattern.

In another embodiment, the base 12 can be micro-patterned or nano-patterned with one or more additional patterns with different surface chemistries. In this embodiment, the mobile layer contains a plurality of sets of adsorbing particles 18, each set with chemistry specific for adsorption to one of the patterns. As a result, the device can contain additional visible or machine-readable patterns and data, such as a bar code or encrypted information that is only detectable with the use of a machine.

Each of the layers will now be described in more detail. The base 12 provides support and partial containment for the mobile component. Preferably, the base is sufficiently flexible to be depressed by the pressure of a finger, thereby adding to the energy of the closed thermodynamic system to cause desorption of the adsorbable particles 18 from the surface of the pattern. Preferably, the base is strong enough to avoid tearing and degradation over time from handling, sunlight, and washing.

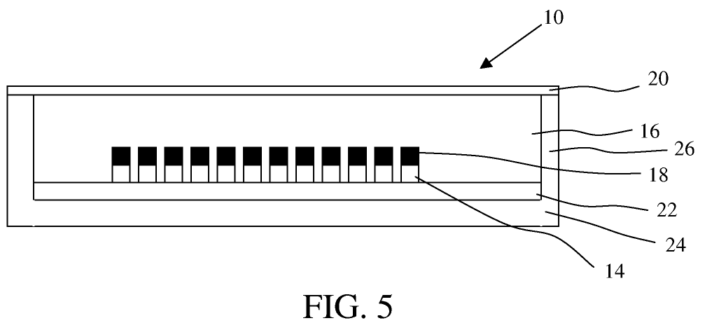

As shown in FIG. 5, the base 12 may have multiple layers. The base has a substrate 22 with the pattern 14 formed thereon. In an exemplary embodiment of the present invention, the substrate is a multi-layer semiconductor chip. For example, the semiconductor chip may have a GaAs surface, upon which a layer of Si₃N₄ has been deposited to form the pattern 14. The Si₃N₄ can be micro-patterned or nano-patterned using etching and deposition techniques known in the semiconductor industry. Accordingly, the accompanying adsorbable particles have chemistry such that the particles will adsorb to Si₃N₄ but not to GaAs.

Additionally, the substrate may be a plastic with a pattern of oligonucleotides, antibodies or antigens bound thereto. Accordingly, the accompanying adsorbable particles are epitopes or homologous oligonucleotides labeled with fluorophores or other color-generating agents.

Preferably the substrate has a thickness from about 5 to about 100 micrometers, thereby allowing items as thin as a Federal Reserve Note or other paper product to be labeled. As will be understood by those skilled in the art, the choice of materials for the substrate and the pattern can be widely varied depending on the mobile component and adsorbable particles used in the device.

In an additional embodiment, the base 12 or substrate 22, may be semiconductor material containing multiple patterns that also form microcircuits. By continuing these microcircuits through the walls of the device and connecting to an electrically active integrated circuit system, adsorption resulting from charged surface (or other electromagnetic) phenomena may be incorporated into the device. As a result, multiple patterns may be formed via microcircuit switching processes in a manner known to those in the semiconductor industry. Additionally, if the base 12 is optically clear, then the pattern formed by adsorption may be visible on both sides of the device.

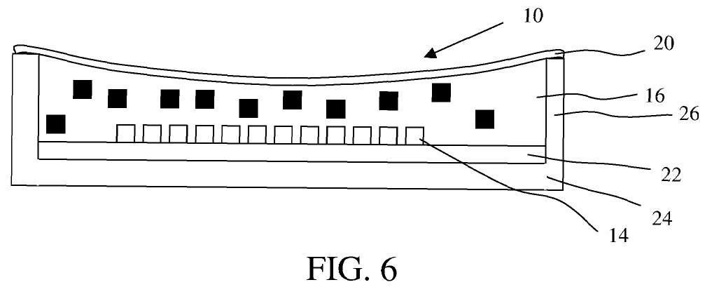

Optionally, as shown in FIGS. 5 and 6, the base 12 has a protective layer 24 coupled to a non-patterned side of the substrate 22. The protective layer 24 may allow the system to be compressed to a greater degree which, in turn, results in more energy being input into the closed system. The protective layer 24 can be made of, for example, silicone rubber or silicone elastomer, as well as other materials capable of fabrication at a scale commensurate with the desired size of the device.

The protective layer 24 may be coupled to the substrate 22 using, for example, an adhesive, chemical, thermal or ultrasonic welding. Additionally, the substrate can be deposited directly onto the protective layer, such as through, for example, printing, sputter coating or spin coating. The pattern material may be subsequently formed by etching a fully deposited layer or depositing the pattern material only in preselected areas of the surface of the substrate 22.

Silicone rubbers and elastomers are routinely produced at a commercial thickness of 0.005 inches, and fabrication of these materials to a lower thickness is achievable. Known techniques, including micro imprinting lithography, soft lithography, direct deposition, three dimensional printing, and laser stereolithography, can be used for fabricating sub-micrometer structures from polymeric and elastomeric materials. For example, see Y. Lu and S. C. Chen, Micro and nanofabrication of biodegradable polymers for drug delivery, Advanced Drug Delivery Reviews (56):1621-1633, 2004 (Elsevier), the entire contents of which are hereby incorporated herein by reference. The thickness of the protective layer is preferably from about 5 micrometers to about 100 micrometers, and more preferably from about 20 to about 50 micrometers. Because the base 12 does not adsorb the particles 18, the base 12 creates the contrast necessary for pattern 14 to create an image when the particles 18 are adsorbed to the pattern 14.

Preferably, the base 12 has a lip 26 around an outer edge to hold the mobile component 16 adjacent the base and to support the cover 20. The lip 26 may be formed from the material of the protective layer 24. Alternatively, a solid spacer (not shown) is inserted between the base 12 and the cover 20 to allow placement of the mobile component 16 between the base 12 and the cover 20. Alternatively, a spacer is formed on the base 12 by etching, deposition or other known fabrication method.

The lip forms a functional reservoir to hold the mobile component 16 so that the adsorbing particles 18 are constantly making contact with the complementary chemistry of the adsorbing pattern 14 via random thermal motion. By adjusting the concentration of adsorbing particles 18, the chemical composition of the mobile layer 16, the pattern surface area, and the total volume of the reservoir, the rate of adsorption-based molecular self-assembly and the speed of ADR cycling may be controlled. The sides 22 and the cover 20 are annealed via adhesive or other method so as to withstand the compression associated with multiple ADR cycles.

For use as a windowed feature in a Federal Reserve Note or other applications such as driver's licenses and ID cards made of paper or plastic material with a width in the range of 100 to 120 micrometers, the lip extends out from the base 12 from about 20 to about 100 micrometers, and more preferably from about 40 to about 80 micrometers.

The pattern can be formed on the base using surface derivatization. Patterning may utilize nanopatterning and micropatterning techniques used in circuit design and biotechnology as will be further discussed below. The size of the pattern is preferably visible to the unaided human eye. For example, a convenient visible pattern size for a windowed feature in a Federal Reserve Note, driver's license, or ID card may occupy about one half or more of a windowed area of about 1 cm×about 1 cm.

The pattern itself is formed by the alignment of a series of micro-patterned or nanopatterned geometric regions on the substrate. For example, a visible line with the dimensions of 1 millimeter in width and extending 1 cm in length may be formed by alternate spacing of 500 micropatterned lines 1 micrometer in width×1 centimeter in length of adsorbing surface interspersed with 500 lines of substrate material 1 micrometer in width×1 centimeter in length. The ratio and specific orientation of adsorbing and non-adsorbing material is determined by, for example, the desired level of contrast, color, and brightness associated with the detectable pattern. Preferably, the pattern is not detectable in a customary way, such as by an unaided human eye, without adsorption of the adsorbable particles.

In addition, the base may contain one or more additional patterns (not shown) which are detectable only using a machine reader (and to which the adsorbable particles do not attach). Such patterns may be formed using processes similar to those used to form the original pattern.

The mobile component 16 can be an aqueous solution containing the adsorbable particles 18. The solution may also contain a nonaqueous solvent, detergent, or other agent, to modify the free energy of adsorption. Additionally, the solution may contain antioxidants or other preservatives to prolong the life of the chosen color-generating agent. Additionally, the solution may contain elements to modify viscosity, which may in turn control the rate at which the adsorbable particles undergo ADR cycling.

The adsorbable particles can be, for example, a luminescent material such as a fluorophore, or a coloring agent such as a hydrophilic dye. Additionally, the adsorbable particles can include, for example, a dyed linked oligonucleotide for binding to a complementary oligonucleotide bound to the pattern 14 on the base 12. Additionally, the adsorbable particles can include an antibody with the pattern having the corresponding antigen, or vice versa.

Importantly, the adsorbable particles 18 are mobile when suspended between the base 12 and the cover 20 such that adsorption and desorption can occur. Preferably, the mobile component 16 is selected so that the adsorbable particles 18 are mobile and adsorbable to the pattern 14 in temperatures ranging from about −20 to about 70 degrees Celsius. The mobile component 16 can also be a gel or solid material that releases the adsorbing agent upon application of pressure or input of other forms of energy to the closed thermodynamic system.

The adsorbable particles 18 have two functions. The first function is reversible adsorption to the pattern on the base. The second function of the adsorbable particles is to interact with visible or other types of excitation light so that upon adsorption and formation of the patterned image, the pattern may be quickly and easily seen by the human eye or another detector. The adsorbable particle may be labeled with a detectable label. Additionally, the adsorbable particle may itself be detectable.

Chromogenic dyes, such as malachite green, bromothymol blue, and analine derivatives may be linked to the adsorbable particle. Other small-molecule colored dyes can be used where the dyes have a functional linking chemistry that allows attachment to the adsorbable particle without disrupting the efficacy of the adsorbable particle or the dye.

Conventional dye molecules produce their effect by absorbing or scattering light. Although the effect of individual dye molecules can be small, significant changes in at least one visual parameter are obtained upon assembly of a macroscopic pattern. Additionally, assembly of a macroscopic structure may cause detectable changes if the dyes are in close physical proximity due to quenching or energy transfer effects.

Additionally, luminescent, phosphorescent, and fluorescent dyes can be used as detectable labels. Many known fluorescent chromophores absorb ambient light provided by normal forms of illumination (sunlight, incandescent or fluorescent bulbs) and emit at wavelengths in the visible spectrum. The advantage of using luminescent, phosphorescent, and fluorescent labels is that the emitted light is at a different wavelength from the excitation and background light, thereby providing an acceptable signal-to-noise ratio over the background. Preferably, the dyes and chromophores are chosen, and the pattern for adsorbable particles arranged, to minimize: 1) shadowing of deeper molecules by surface molecules, 2) dye-dye interactions, and 3) fluorescence resonance energy transfer (FRET).

Preferably, the concentration is at least about 1000 dye molecules per square micrometer of pattern for visualization by a human eye. More preferably, the dye concentration is between about 10,000 and 30,000 dye molecules per square micrometer of pattern.

Examples of fluorescent/luminescent dyes useful for human detection using visible light include Alexa Fluor® 488 and Alexa Fluor® 555 by Invitrogen Corporation, 1600 Faraday Avenue, Carlsbad, Calif. 92008. Examples of phosphorescent dyes for human detection using visible light include particulate metals used in signage, such as Glowbug Pigments by Capricorn Chemicals, Lisle Lane, Ely, Cabs CB7 4AS United Kingdom.

Additionally, the label may be a quantum dot such as those manufactured by Invitrogen Corporation, 1600 Faraday Avenue, Carlsbad, Calif. 92008 and by Evident Technologies, 216 River Street, Suite 200, Troy, New York 12180. Additionally, the label can be a small metal colloid, micro-particulate or nano-particulate metal displaying color generating, or reflective properties, such as gold and copper. Iron-based ferromagnetic micro-particles or nano-particles may also be usable.

The cover 20 is preferably translucent and more preferably substantially transparent to allow for viewing of the adsorbable particles. For applications where the depth of the device is limited, such as for incorporation into a windowed security feature for paper currency, the cover thickness may range from about 5 to about 100 micrometers, and more preferably range from about 5 to about 15 micrometers.

The cover 20 can be made of a polymer such as linear high density polypropylene (LHDP), polyethylene, polycarbonate and polymethylmethacrylate. Preferably, the cover 20 is made of Kapton® polyimide film which is supplied commercially by DuPont™, Wilmington Del., as a film having a 7.5 micrometer thickness. Additionally, the cover 20 can be made of other flexible clear materials, such as Tefzel® fluropolymer film which is supplied commercially by DuPont™, as an optically clear film having a 12.7 micrometer thickness.

The behavior of the device is partially controlled by the properties of the cover. The mobile component is preferably relatively incompressible. This helps reduce the possibility of the cover being cracked or damaged during the compression cycle. The physical parameters of the materials of the cover and the protective layer (if present), such as the elastic deformability, Youngs Modulus, and toughness affect how energy is transferred into the device during compression. The cover material contributes to the speed with which the cover ‘snaps back’ after compression.

The primary driving force for desorption is assumed to be the fluid dynamics, especially the increased thermal energy of individual molecules in solution and turbulence resulting from hydrodynamic fluid motion. Both these effects are created by compression. However, if the ‘snap-back’ is rapid enough a small vacuum may form over the liquid creating a brief period of cavitation before the device regains its original shape. This cavitation may further desorption.

Additionally, if the adsorbent particles are suspended in an aqueous solution, a hydrophobic cover may enhance cavitation and generally enhance product performance and lifetime due to a lack of interaction with both the aqueous solvent and hydrophilic adsorption particles. Hydrophobic behavior is expected for materials such as Tefzel®. In a preferred embodiment, an adhesive is used to couple the cover to the base.

In use, a human or machine recognizable image is formed upon adsorption of the adsorbable particles to the pattern on the base. Application of pressure disrupts the visible image. Release of the pressure results in rapid spontaneous reassembly of the image. The cycle of assembly, disassembly, and reassembly should be repeatable unless the physical integrity of the device is destroyed.

The dyes used are optimized for the usage of the device. For example, where the device is used as a security feature in currency, preferably, the dyes are selected for visibility in the green range of visible light, because of the inherent efficiency of human color vision. Other considerations such as contrast and the background color of the item into which the device will be incorporated will affect the choice of dye, chromophore or other color and contrast generating agent. For many applications, the time for adsorption and desorption should fall within that which is optimum for human visual acuity and cognition. This time is preferably from about ½ second to about 5 seconds to provide a quick check of authenticity, but not be so fast as to be undetectable. The amount of pressure needed for disruption is preferably capable of being induced by a human hand without the need for a specialized instrument or ‘reader’. The number of assembly, disassembly, and reassembly cycles that the device can go through is preferably greater than 1000 and more preferably greater than 100,000.

The device may be usable in currency, and therefore will have a width and length ranging from about 1 millimeter to several centimeters and a depth of from about 50 micrometers to about 100 micrometers. In additional applications, the size is variable and only limited by the size necessary for detection by whatever detection apparatus is employed.

Other variables of the device, such as the pressure sensitivity, are customizable for specific applications. For example, it may be useful to have the adsorbing particle be a fluorescent dye that chemically degrades after a defined amount of time. It may also be useful to have multiple patterns and multiple adsorbing species, some of which emit light or other types of signals not directly visible to the human eye but that are machine-readable.

The device is preferably integrated into the product to be protected so that removal of the device renders the device inoperable. For example, in the case of hard goods, the device is preferably attached using an adhesive. In the case of clothing, garments, and paper currency, the device is preferably interwoven with the fibers of these items with further use of adhesive materials.

In an additional embodiment of the present invention, multiple devices can be sandwiched together to create a three dimensional device. Depending on the composition of the individual devices and the manner in which they are arranged in three dimensions, it may be possible to generate holographic or motion effects by tilting or otherwise altering the angle at which the device is viewed. If the pattern has been fabricated from semiconductor or other electronically active materials and continued through the body of the device then dynamic effects may be achieved via the input of electrical or electronic energy as previously described.

Pattern Formation

The substrate 22 may be fabricated using standard semiconductor processing procedures such as polished (100) oriented undoped GaAs wafers. The pattern 14 may be formed on the substrate 22 by deposition of common insulators, such as amorphous Si₃N₄ and SiO₂ in films deposited through plasma-enhanced chemical vapor deposition (PECVD) on the substrates.

Photolithography may be used to produce micrometer length patterns, and dry etching of Si₃N₄ and SiO₂ may be accomplished with CF₄ and CHF₃, respectively, to reveal the underlying substrate. Photolithography may likewise be used to define patterns for deposition, with subsequent liftoff of deposited metals: Au, Pd, Pt, Ti, and Al using e-beam or thermal evaporation. The patterned substrate can be exposed to an oxygen plasma etch as a final dry cleaning to remove organic residues.

Patterned substrates may also be made from molecular beam-epitaxy (MBE) wafers of layered GaAs and AlGaAs, with the AlGaAs layer exposed by using an etch of H₂O₂/NH₄OH.

In an additional embodiment of the present invention, the patterned base is formed using elastomeric stamping technology, such as that taught by Colin D. Bain, E. Barry Troughton, Yu Tai Tao, Joseph Evall, George M. Whitesides, and Ralph G. Nuzzo, Formation of monolayer films by the spontaneous assembly of organic thiols from solution onto gold, J. Am. Chem. Soc. 111:321-335 (1989), the entire contents of which are hereby incorporated herein by reference.

As taught by Bain et al., a 1 to 5 nm film of titanium is evaporated onto a glass coverslip or silicon wafer to promote adhesion of gold to the surface. A 10-200 nm film of gold is then evaporated onto the surface. The resulting gold surface can then be patterned by selectively applying a solution of ethanolic alkylthiol. Mixed monolayers may be formed if the ethanolic solution of T-functionalized alkylthiols contains two or more different thiols.

The pattern can be produced using lithiography, such as taught in Xia, Y.; Whitesides, G. M. AR Mater. Res. 1998, 28, 153, Soft Lithiography, the entire contents of which are hereby incorporated herein by reference. One lithiography method that can be used is microcontact printing. A stamp with a patterned relief is formed from elastomers, such as poly(dimethylsiloxane), PDMS or polymethylmethacrylate (PMMA), that have been poured over a master, cured and then peeled. The masters are manufactured from photolithography, e-beam writing, micromachining or relief structures etched into metals. Each master may be used to produce up to 50 stamps, and each stamp may be used multiple times.

The stamp is inked with an ethanolic solution of T-functionalized thiol and brought into contact with the gold surface for 10-20 seconds resulting in a gold thiolate monolayer at the areas of contact. One specific method uses a stamp replicated from a photolithographically-patterned polymethlymethacrylate master capable of transferring thiols to a gold surface.

Patterns may be further formed on this surface using combinations of hydrophilic HS(CH₂)₁₅COOH and hydrophobic HS(CH₂)₁₅CH₃ self-assembly-forming compounds. A hydrophobic dye in a solution containing water can be used to selectively adsorb to the resulting hydrophilic patterned areas.

Method of Manufacture

Once the pattern 14 has been fabricated onto the substrate 22, as described above, the non-patterned side of the substrate 22 is sealed onto the protective layer 24 using a silicone adhesive. Following sealing of the substrate 22 to the protective layer 24, the cover 20 is sealed to the lip 26 using an adhesive, such as a silicone adhesive. Alternatively, the cover is sealed to the lip 26 using chemical, ultrasonic or thermal welding. The mobile component 16 is then pumped into the space between the substrate 22 of the base and the cover.

Pumping of the mobile component may be done using two micropipettes connected to a reservoir of mobile component, and a vacuum system respectively, the micropipettes being inserted through the lip or cover. Once the device is filled with mobile component, the pipettes are withdrawn and the penetration points in the lip or cover sealed via local application of heat or via application of a sealant.

### EXAMPLE

The example below is for illustrative purposes only. As will be understood by those skilled in the art, the size of the device may vary by application and the dimensions and components described herein are by way of example only and are not intended to limit the scope of possible sizes and components.

The exemplary device uses a fabrication method based on semiconductor technology to create a device fitting into a clear plastic window feature of a standard United States Federal Reserve Note. To fit in the United States Federal Reserve Note, the device preferably has physical dimensions of about 1 centimeter×about 1 centimeter×about 109 micrometers (length×width×depth).

The base has a substrate formed of GaAs with a pattern of Si₃N₄ formed thereon. The thickness of the substrate is from about 10 micrometers to about 30 micrometers. The base is further encased by a protective layer formed from a single piece of silicone elastomer having a thickness of from about 10 micrometers to about 30 micrometers. A portion of the protective layer is formed as the lip of the device. The lip will partially contain the mobile component. The lip extends out from the remainder of the protective layer from about 60 to about 70 micrometers beyond the plane of the pattern. The side of the GaAs substrate facing the protective layer is sealed to the protective layer using a silicone adhesive or other known method.

A cover of DuPont™ Kapton® polyimide film having a 7.5 micrometer thickness is sealed to the lip formed by the protective layer. The total thickness of the device with the cover sealed to the lip is less than about 109 micrometers.

The mobile component is a solution with a 0.4 micromolar to 4.0 micromolar concentration of 8- to 10-mers of polylysine end-labeled with Fluorescin dye for selective adsorption to the Si₃N₄ nano-pattern. A detailed discussion of the selective adsorption of polylysine to a Si₃N₄ pattern on a GaAs background is found in an article entitled Differential adhesion of amino acids to inorganic surfaces by R. L. Willett et al., Proc. Nat. Acad. (USA):102(22), p. 7817-7822, 2005, the entire contents of which are hereby incorporated herein by reference.

Because the dye-labeled polylysine may be washed off the Si₃N₄ surface with appropriate solutions, reversible adsorption may be assumed. Adsorption energetics may be modified by varying the solvent composition of the mobile layer. Given the data produced by Willett et al., it is assumed that adsorption of such molecules occurs at 20,000 molecules per square micrometer of Si₃N₄ surface. With a substrate thickness of 30 micrometers and a protective layer thickness of 30 micrometers and a cover thickness of 10 micrometers thickness including sealant for a total of 70 micrometers of occupied space. Given the total thickness of the Federal Reserve Note is 109 micrometers, 39 micrometers of depth is available for the mobile phase. With length and width dimensions of about 1 centimeter each, the device has about 4 microliters of volume for the mobile component.

Assuming an adsorption coverage of 20,000 dye-tagged oligopeptides per square micrometer of Si₃N₄, and further assuming that only 50% of the substrate surface is patterned with Si₃N₄; then a concentration of 0.4 micromolar adsorbent solution contains enough molecules to fully cover the Si₃N₄ surface. This assumes 100% of the adsorbent molecules in solution are adsorbed. By raising the concentration of adsorbent from 0.4 micromolar to 4.0 micromolar, full coverage may be achieved with only 10% adsorption. Biomolecules such as an 8- or 10-mer of polylysine containing a fluorescent dye tag, such as fluorescin, are expected to be soluble at 4.0 micromolar concentrations and even higher.

### CONCLUSION

The present invention uses micro-fabrication or nanofabrication to create a reversible molecular self-assembling system that is a closed thermodynamic system capable of exchanging energy but not matter with its environment. The input or loss of relatively small amounts of energy will cause the system to change states from assembled to disassembled or the reverse. Authentication is proven by the dynamic behavior of the assembly, disassembly, and reassembly cycling.

In its simplest form, an image is formed within a product tag by the self-assembly of particles onto a pattern. The molecules have a specific binding affinity for the chemistry of the pattern. Preferably, the molecules have the ability to fluoresce in the visible spectrum under conditions of ambient light. As a result of stable binding to the surface, the generally coherent light emitted by the assembled structure forms a macroscopic visual image.

When this structure is perturbed by the input of a small amount of thermal energy and hydrodynamic turbulence, such as by pressing a thumb down on the tag, the image inside the tag literally disappears in front of the user. As heat energy and fluid turbulence spontaneously dissipate, the molecules undergo a new cycle of self-assembly on the pattern and the macroscopic image reappears. The device is integrated into the product in such a manner that any attempt to physically alter or remove it from its original location either destroys or distorts the ADR property to an extent that makes such tampering obvious.

The device of the present invention has many advantages. Due do its technical complexity and the equipment necessary to produce the device, the device of the present invention cannot be easily counterfeited. The present invention creates dynamic behavior via cycles of molecular self-assembly. Once fabricated, the device may operate indefinitely driven only by its internal physiochemical structure, and the input of simple physical energy.

Unlike radio frequency identification devices or other identification devices that produce or require some type of active signal as part of their authentication algorithm, the device of the present invention is preferably designed to be activated and detected by unaided human beings under the normal range of environmental light conditions, from low incandescent up to full sunlight. This allows for product verification at any time in any location without additional enabling technology or devices. This makes the device of the present invention appropriate for use in many different products, such as currency and a wide range of consumer goods.

However, the device may also have covert signal generating systems that require instrumentation or special training for detection, such as fluorescent, infrared, electromagnetic, and electro-optical labels attached to the adsorbable particles. Additionally, selected molecular components of the macroscopic image can develop a secondary cryptic pattern for added security.

Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts and drawings, and all the steps in any method or process disclosed, may be combined in any combination except combination where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. §112.

What is claimed is:

1. A security device comprising:
a base having a pattern thereon;
a mobile component disposed in contact with the base, the mobile component containing a plurality of reversibly adsorbable particles; and
a cover attached to the base around the mobile component to contain the mobile component in contact with the base;
wherein the adsorbable particles are mobile and reversibly changeable between a first state where the adsorbable particles are adsorbed to at least a predetermined percentage of the pattern and a second state where the adsorbable particles are adsorbed to less than the predetermined percentage of the pattern.

2. The security device of claim 1 wherein when the adsorbable particles are in the first state, the pattern is visible to an unaided human eye.

3. The security device of claim 2 wherein the base further comprises a second pattern that is not visible to an unaided human eye when the adsorbable particles are in the first state.

4. The security device of claim 2 wherein the base further comprises a second pattern that is not visible to an unaided human eye.

5. The security device of claim 2 wherein the adsorbable particles comprise a dye.

6. The security device of claim 2 wherein the base further comprises:
a protective layer; and
a substrate attached to the protective layer, the pattern being formed on the substrate.

7. The security device of claim 6 wherein the protective layer comprises at least one of the group consisting of silicone rubber and silicone elastomer.

8. The security device of claim 7 wherein the cover further comprises at least one of the group consisting of a polyimide film and a fluoropolymer film.

9. The security device of claim 1 wherein:
the pattern is not visible to an unaided human eye when the adsorbable particles are in the first state; and
the pattern is visible to an unaided human eye when the adsorbable particles are in the second state.

10. The security device of claim 1 wherein the adsorbable particles change from the first state to the second state and back to the first state in less than 5 seconds.

11. The security device of claim 1 wherein the device is operable in a temperature range of from about −20 degrees Celsius to about 70 degrees Celsius.

12. The security device of claim 1 wherein the adsorbable particles can cycle from the first state to the second state and back to the first state more than 10,000 times.

13. The security device of claim 1 wherein the base comprises a lip around an outer edge; and the cover is attached to the lip.

14. A method for making a security device comprising:
forming a base with a pattern;
coupling the base to a cover;
injecting a mobile component between the base and the pattern through the cover, the mobile component comprising a plurality of adsorbable particles; and
sealing the cover;
wherein the adsorbable particles are mobile and reversibly changeable between a first state where the adsorbable particles are adsorbed to at least a predetermined percentage of the pattern and a second state where the adsorbable particles are adsorbed to less than the predetermined percentage the pattern.

15. The method of claim 14 wherein forming a base comprises:
depositing a pattern material on a substrate in a pattern; and
attaching the substrate to a protective layer.
